(19) United States
(12) Patent Application Publication
(10) Pub. No.: US 2020/0346929 A1
(43) Pub. Date: Nov. 5, 2020

(54) Methods and Systems for Producing Biochar

(71) Applicant: Johnny Keith Cagigas, Advance, NC (US)

(72) Inventor: Johnny Keith Cagigas, Advance, NC (US)

(21) Appl. No.: 16/864,612

(22) Filed: May 1, 2020

Related U.S. Application Data
(60) Provisional application No. 62/842,785, filed on May 3, 2019.
(60) Provisional application No. 62/961,922, filed on Jan. 16, 2020.

(51) Int. Cl. C01B 32/05; C05D 9/00; C09K 17/02; A61L 9/014; B01J 20/20; B01J 20/30; A23K 20/20; C10B 53/02; C10B 1/10; B01D 53/14

(52) U.S. Cl. CPC C01B 32/05; C05D 9/00; C09K 17/02; A61L 9/014; B01J 20/20; A61L 2209/22; A23K 20/20; C10B 53/02; C10B 1/10; B01D 53/14; B01J 20/3078

(57) ABSTRACT

In various embodiments, a method may comprise disposing biomass into a processing chamber; regulating at least one of a temperature and a pressure in the processing chamber; and/or processing the biomass to create biochar. The biomass may comprise cellulose acetate.

### CROSS-REFERENCE TO RELATED APPLICATIONS

This application is a Non-Provisional of, and claims priority to and the benefit of, U.S. Provisional Patent Application No. 62/842,785, filed May 3, 2019 and entitled “METHODS AND SYSTEMS TO THERMALLY DECOMPOSE BIOMASS TO PRODUCE BIOCHAR,” and U.S. Provisional Patent Application No. 62/961,922, filed Jan. 16, 2020 and entitled “METHODS AND SYSTEMS TO THERMALLY DECOMPOSE BIOMASS TO PRODUCE BIOCHAR,” which are hereby incorporated by reference herein.

### BACKGROUND

Various materials produced industrially or commercially are discarded in ways that may be detrimental to the environment. For example, many materials are disposed of in landfills as waste. As a specific example, over eight hundred thousand tons of cellulose acetate (e.g., synthetic fiber cellulose acetate, from a particular product(s) or industry(s)) end up in landfill each year, along with thousands of additional tons that are improperly discarded as litter. Such cellulose acetate waste may come from cigarettes and/or the cigarette industry (e.g., from the filters of used cigarettes (4.5 trillion cigarettes are thrown away every year), from cigarette or cigarette filter manufacturers (e.g., filters that do not meet specifications), and/or the like). Cellulose acetate, especially synthetic cellulose acetate fibers (a polymeric material) used in cigarette filters, can take ten years or longer to degrade or decompose. In fact, synthetic cellulose acetate fibers, on average, may only lose 37.8 percent of their initial mass after two years of decomposition. Therefore, disposal of such materials in landfill or as litter can result in their presence and pollution of the surrounding area for years.

### SUMMARY OF THE DISCLOSURE

The present disclosure relates to methods, controlled process conditions, and systems to thermally decompose biomass, which may include but is not limited to wood, cellulose acetate and/or other cellulosic materials, organic materials, agriculture crops, hemp, cannabis, marijuana, byproducts of any of the foregoing, and/or any other suitable material, to make biochar. The biomass and/or biochar may be processed using carefully managed process conditions within a biochar reactor with approximately zero or other low ranges of oxygen in the processing atmosphere. The temperature may be changed and/or maintained in a controlled process with a dwell time as determined by the biomass and the desired properties of the resulting biochar. The biochar may require screening, pressing, and/or additional sizing to reach the desired particle size and/or consistency for the application. Additionally, various biomass materials may be introduced or combined (e.g., with cellulose acetate) to achieve required or desired physical and chemical properties.

The biochar may be utilized in many capacities, such as a soil supplement, fertilizer, odor-reduction media, livestock feed supplement, and may have an additional benefit of reducing CO₂ within the environment. In various embodiments, a method of forming biochar also purposes as an alternative to landfill disposal. The biochar may have the benefits of increasing and improving agricultural output, reducing odor in poultry, bovine, and/or swine operations, and/or potentially reducing soil contamination. A further benefit of the biochar may result from the absorption and/or adsorption properties of the biochar when added to the soil and used to treat water to reduce harmful effects related to fertilizers and contaminants. The resultant products from forming biochar in accordance with embodiments discussed herein of syngas, oils, etc. may be converted into power, insecticides, or other commodities. Additionally, the process of forming the biochar may be environmentally neutral or positive, and/or a carbon-neutral or carbon-negative solution.

In various embodiments, a method may comprise disposing biomass into a processing chamber; regulating at least one of a temperature and a pressure in the processing chamber; and/or processing the biomass to create biochar. The biomass may comprise cellulose acetate. In various embodiments, the processing chamber may comprise a kiln. In various embodiments, the processing chamber may comprise a rotary kiln. In various embodiments, an atmosphere in the processing chamber during the processing the biomass may comprise little or no oxygen. In various embodiments, the atmosphere in the processing chamber may comprise a nonreactive gas. In various embodiments, the biomass may comprise between 50 and 100 percent by weight cellulose acetate. In various embodiments, the cellulose acetate may comprise a synthetic fiber cellulose acetate.

In various embodiments, the method may further comprise procuring the biomass comprising the synthetic fiber cellulose acetate from discarded cigarette filters and/or discarded cigarette filter components. In various embodiments, the regulating the temperature comprises increasing the temperature in the processing chamber 10 degrees C. per minute up to between 550 and 800 degrees C. In various embodiments, the method may further comprise sizing the biomass prior to disposing the biomass into the processing chamber. In various embodiments, sizing the biomass may be completed via a shredder. In various embodiments, the biomass may be disposed into the processing chamber from a feed system comprising a feeder valve.

In various embodiments, the method may further comprise discharging an exhaust from the processing chamber through an exhaust system; and/or scrubbing the exhaust in a scrubber comprised in the exhaust system. In various embodiments, the method may further comprise discharging the biochar into a discharge system; and/or cooling the biochar. In various embodiments, the method may further comprise supplementing the biomass comprising cellulose acetate with biomass comprising at least one of animal manure or a plant-originating material. In various embodiments, the method may further comprise utilizing the biochar as at least one of a soil supplement, a fertilizer, an animal feed supplement, or an odor reducer.

In various embodiments, a biochar may be formed by pyrolyzing biomass comprising cellulose acetate. In various embodiments, the cellulose acetate may comprise synthetic fiber cellulose acetate. In various embodiments, the biomass may comprise between 50 and 100 percent by weight cellulose acetate. In various embodiments, the biomass may further comprise at least one of animal manure or a plant-originating material.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures.

FIG. 1 illustrates a system for creating biochar, in accordance with various embodiments; and

FIG. 2 illustrates a flow diagram of a method for creating biochar, in accordance with various embodiments.

### DETAILED DESCRIPTION

All ranges may include the upper and lower values, and all ranges and ratio limits disclosed herein may be combined. It is to be understood that unless specifically stated otherwise, references to “a,” “an,” and/or “the” may include one or more than one and that reference to an item in the singular may also include the item in the plural.

The detailed description of various embodiments herein makes reference to the accompanying drawings, which show various embodiments by way of illustration. While these various embodiments are described in sufficient detail to enable those skilled in the art to practice the disclosure, it should be understood that other embodiments may be realized and that logical, chemical, and mechanical changes may be made without departing from the scope of the disclosure. Thus, the detailed description herein is presented for purposes of illustration only and not of limitation. For example, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, or the like may include permanent, removable, temporary, partial, full, and/or any other possible attachment option. Additionally, any reference to without contact (or similar phrases) may also include reduced contact or minimal contact.

The present disclosure relates to methods, controlled process conditions, and systems to process (e.g., thermally decompose) biomass (e.g., comprising cellulose acetate and/or other cellulosic biomass) to produce biochar. Biochar is a charcoal-like, carbonaceous material comprising a great amount of surface area due to its porous form. These characteristics of biochar allow it to capture and hold particles, moisture, nutrients, materials, microorganisms, and/or the like.

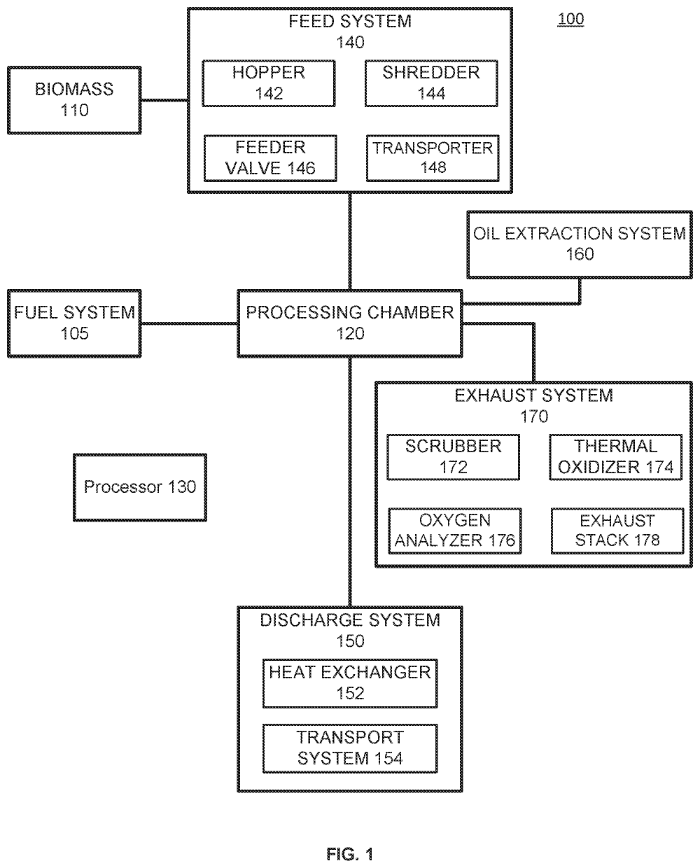

With reference to FIG. 1 illustrating a system 100 for creating biochar, biomass 110 may include wood, cellulose acetate and/or other cellulosic biomass, organic materials, agriculture crops, hemp, cannabis, marijuana, portions or byproducts of any of the foregoing, and/or the like. Cellulose acetate used in producing biochar in accordance with various embodiments of this disclosure may comprise synthetic fiber cellulose acetate intended or made for use in cigarette filters. For example, the cellulose acetate comprised in the biomass may be from cigarette filters, cigarette butts, filter rods, and/or any other cigarette filter component, or from manufacturers of cigarettes or cigarette components, such as discarded cellulose acetate (e.g., that did not meet specifications for actual use in a cigarette filter), cellulose acetate tow, and/or the like. The cellulose acetate may be procured from any suitable source, such as landfill or other garbage location or receptacle, litter, cigarette component manufacturers, and/or discarded, used, defective, or unwanted cigarette filters or cigarette filter components. Accordingly, the cellulose acetate used to make the biochar discussed in this disclosure may be cellulose acetate that otherwise would be discarded and remain in landfill (or other garbage disposal area) or as litter. In various embodiments, the biomass used in producing biochar in accordance with various embodiments of this disclosure may comprise between 20 and 100 weight percent cellulose acetate, between 30 and 100 weight percent cellulose acetate, between 50 and 100 weight percent cellulose acetate, between 70 and 100 weight percent cellulose acetate, between 80 and 100 weight percent cellulose acetate, between 85 and 95 weight percent cellulose acetate, and/or any other suitable amount of cellulose acetate.

In various embodiments, system 100 may comprise a fuel system 105, a processing chamber 120, a processor 130, a feed system 140, a discharge system 150, an oil extraction system 160, and/or an exhaust system 170. Any of the components of system 100 may be in electronic, mechanical, and/or fluid communication with one another, and/or coupled to one another. In various embodiments, at least some components of system 100 may be arranged relative to one another such that a material may pass between them (e.g., biomass, resulting biochar, byproducts, etc.). In various embodiments, a system for producing biochar may comprise any suitable number of each component or subcomponent (e.g., multiple feed systems 140, transporters 148, processing chambers 120, and/or the like).

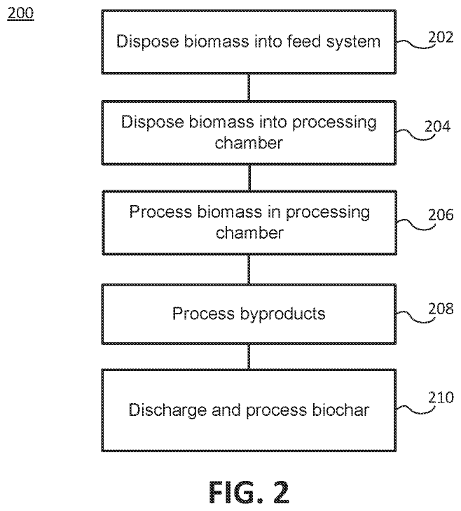

In various embodiments, with reference to FIGS. 1 and 2, a method 200 for creating biochar may comprise disposing biomass 110 into feed system 140 (step 202). In various embodiments, a feed system 140 may comprise a hopper 142, a shredder 144, a feeder valve 146, and/or a transporter 148. Biomass 110 may be disposed into hopper 142 and/or shredder 144 as part of step 202. Shredder 144 may shred and/or otherwise size biomass 110 into a desired shape and/or size. Hopper 142 may receive, store, and/or feed biomass 110 into another component of feed system 140 and/or processing chamber 120. For example, hopper 142 may receive biomass 110 and feed biomass 110 into shredder 144 and/or feeder valve 146. As another example, hopper 142 may receive biomass 110 from shredder 144 and/or feeder valve 146. In various embodiments, hopper 142, shredder 144, and/or feeder valve 146 may be coupled to one another in any suitable order or arrangement.

In various embodiments, feeder valve 146 may be an airlock valve (e.g., a knife gate, butterfly valve, double dump valve, a rotary valve, and/or the like). In various embodiments, feeder valve 146 may be coupled to and/or receive biomass 110 from hopper 142 and/or shredder 144, and transfer biomass 110 to processing chamber 120. In various embodiments, feeder valve 146 may be at least partially sealed to the ambient environment to limit air (e.g., oxygen) from entering processing chamber 120.

In various embodiments, a transporter 148 may transport biomass 110 between, and/or may be coupled to or disposed between, hopper 142, shredder 144, feeder valve 146, and/or processing chamber 120. Transporter 148 may be a conveyor (e.g., a screw conveyor, chute conveyor, wheel or roller conveyor, belt conveyor, gravity-fed conveyor, and/or the like), a chute (e.g., a gravity chute), and/or any other suitable device to transport biomass 110 from and/or between hopper 142, shredder 144, feeder valve 146, and/or processing chamber 120. In various embodiments, biomass 110 may pass through hopper 142 and/or shredder 144 and onto a transporter 148 (e.g., a screw conveyor). Transporter 148 may be controlled by a controller (e.g., by a variable-frequency drive). Transporter 148 may transport biomass 110 in a desired amount and/or at a desired rate from and/or between hopper 142, shredder 144, feeder valve 146, and/or processing chamber 120 (e.g., at a rate of a certain number of pounds per minute, or the like). In various embodiments, a transporter 148 may transfer biomass 110 into processing chamber 120, such that biomass 110 is disposed into processing chamber 120 (step 204).

In various embodiments, processing chamber 120 may comprise a transporter similar to transporter 148 to dispose biomass 110 into processing chamber 120. Processing chamber 120 may be configured to allow and/or facilitate the thermal decomposition of biomass 110 to make biochar, for example, from performing a pyrolysis process. In various embodiments, processing chamber 120 may comprise any suitable system to heat, decompose, and/or demanufacture, a material (e.g., biomass 110), such as an oven, a kiln (e.g., an indirect heated rotary kiln), a retort, and/or the like.

In various embodiments, biomass 110 may comprise various materials or combinations thereof to achieve desired physical or chemical properties of the resulting biochar. For example, as discussed herein, biomass 110 may comprise cellulose acetate or other cellulosic biomass, wood, organic materials, manure, agriculture crops, hemp, cannabis, marijuana, portions and/or byproducts of any of the foregoing, and/or the like. Biomass 110 may be processed in processing chamber 120 (step 206). Processing biomass 110 may comprise heating, decomposing, degrading, demanufacturing, and/or similar actions on biomass 110 to create biochar. Processing biomass 110 in processing chamber 120 may include managing and/or changing temperature, pressure, dwell time, and/or other conditions within and/or around processing chamber 120. The atmosphere in processing chamber 120 before, after, and/or during processing biomass 110 may comprise little or no oxygen (e.g., trace amounts of oxygen, such as less than 100, less than 10, or less than 1 parts per million), or any suitable amount of oxygen. In various embodiments, a nonreactive gas (e.g., an inert gas, nitrogen gas, and/or the like) may be provided to and comprised in processing chamber 120 to displace and/or remove oxygen. The nonreactive gas may not contribute to, or may have minimal effect on, any reaction occurring in processing chamber 120 during biomass 110 processing. The nonreactive gas may be supplied to processing chamber 120 by a gas source in fluid communication with processing chamber 120.

In various embodiments, processing chamber 120 may be heated, for example, for biomass 110 processing. A fuel system 105 may be coupled or fluidly coupled to a burner proximate processing chamber 120 or coupled to processing chamber 120. Fuel system 105 may provide a fuel for heating processing chamber 120 such as electricity (e.g., for electrical heating), or natural gas, gasoline, kerosene, oil, and/or any other suitable fuel for burners configured to heat processing chamber 120. In various embodiments, fuel system 105 may provide combustion gas (e.g., oxygen) to facilitate heating of processing chamber 120.

In various embodiments, during processing of biomass 110, the temperature in processing chamber 120 may be increased over a period of time. The temperature in processing chamber 120 may start at between 200 and 400 degrees C., between 250 and 375 degrees C., or about 300 degrees C. (“about” in this context means plus or minus 20 degrees C.). The temperature may be increased in a controlled process, for example, 5 to 10 degrees C. per minute up to between 250 degrees C. and 900 degrees C., or between 600 or 700 or 750 degrees C. and 800 degrees C., with a dwell time (i.e., total processing time), for example, between 20 minutes and 10 hours, 30 minutes and two hours, or 50 minutes and 90 minutes. In various embodiments, the temperature in processing chamber 120 may reach about 700 or 750 degrees with a total dwell time of between 50 minutes and 90 minutes. The temperatures, temperature increases, rates of increase, and/or dwell times during biomass 110 processing may be determined with regard to biomass 110 and its contents, and the desired properties of the resulting biochar. The temperatures in processing chamber 120 during biomass processing to form biochar may be dictated and/or controlled by a processor 130 (which may comprise or be associated with a controller) in electronic communication with processing chamber 120. The processor 130 and/or controller may be in electronic communication with processing chamber 120, fuel system 105, a heating system to heat processing chamber 120, and/or any component of system 100. In various embodiments, system 100 may comprise one processor 130, and/or one or more components of system 100 may comprise and/or be in electronic communication or associated with a processor.

In various embodiments, during processing of the biomass 110, the temperature in processing chamber 120 may be set and maintained at desired setpoints. The temperature may be dictated and/or controlled by processor 130 (as discussed above), which may comprise or be associated with a controller to facilitate temperature changes and/or maintenance. For example, a setpoint of between 500° C. and 800° C., or between 550° C. and 780° C., with a dwell time between 20 or 30 minutes and two hours may be maintained in processing chamber 120.

Processing biomass 110 in processing chamber 120 may produce byproducts and/or exhaust. Such byproducts and/or exhaust may exit processing chamber 120 and enter exhaust system 170. In various embodiments, exhaust (e.g., flue gas) may exit processing chamber 120 and be released into the surrounding environment via an exhaust stack 178. Exhaust stack 178 may be in fluid communication with the interior of processing chamber 120. Byproducts (e.g., off-gasses, particulates, and/or other materials) from biomass 110 processing may be processed (step 208) by exhaust system 170. For example, byproducts may enter a scrubber 172 comprised in exhaust system 170 to remove particulates and/or various chemicals materials (e.g., harmful chemicals or materials). The off-gases may then pass through a thermal oxidizer 174 comprised in exhaust system 170 for combustion. For example, thermal oxidizer 174 may decompose hazardous gases or materials in the byproducts. The combusted byproducts may, in various embodiments, pass through an oxygen analyzer 176 comprised in exhaust system 170 to analyze the amount of oxygen in the byproducts that are exiting system 100 by exiting exhaust stack 178 (e.g., to detect incomplete combustion of byproducts to prevent pollution associated therewith).

In various embodiments, liquid byproducts (e.g., oil) may enter oil extraction system 160. Oil extraction system 160 may be separate from other components of system 100. In various embodiments, oil extraction system 160 may be comprised in and/or in fluid communication with exhaust system 170 or components therein. In various embodiments, gas or liquid byproducts (e.g., an oil) may discharge from scrubber 172 into oil extraction system 160. For example, liquid byproducts may condense in and discharge from scrubber 172 through pumps or other liquid transportation systems to a water cooler (or other cooling device) before reentering scrubber 172. Oil byproduct (e.g., desired oil) may condense and discharge from scrubber 172 and be collected and/or stored in oil extraction system 160.

In various embodiments, biochar resulting from processing biomass 110 in processing chamber 120 may be discharged from processing chamber 120 into discharge system 150 for processing (step 210). Discharge system 150 may comprise a heat exchanger 152 through which the biochar may pass to be cooled (e.g., by water cooling). Heat exchanger 152 may comprise a transport system 154 (e.g., a conveyor, such as a screw conveyor), which may transport the biochar and/or cool the biochar during transport. In various embodiments, transport system 154 may comprise an exit valve, through which the biochar exits discharge system 150. The exit valve may be an airlock valve (e.g., a knife gate, dual knife gates, butterfly valve, double dump valve, a rotary valve, and/or the like) to at least partially prevent oxygen or other ambient air from entering discharge system 150, processing chamber 120, and/or any other component of system 100 in which such gas is unwanted.

In various embodiments, the biochar resulting from processing biomass 110 may be screened, pressed, and/or sized to reach desired shapes and sizes for specific applications of the biochar. The biochar may comprise residual biomass, such as cellulose acetate or other materials discussed herein. Additionally, various biomass materials may be introduced or combined to achieve desired physical and chemical properties of the resulting biochar.

As an example of producing biochar, in accordance with various embodiments, samples of biomass comprising between 97% and 100% by weight cellulose acetate (a synthetic fiber cellulose acetate) were processed at 620 degrees C. for 60 minutes to create Samples A and B of biochar listed in Table 1. The cellulose acetate used in the biomass to create the biochar was cellulose acetate used in, or made for use in, cigarette filters (e.g., from discarded cigarettes and cigarette butts). The biochar resulting from the processed biomass comprised metals, including heavy metals, in amounts shown in Table 1, below.

TABLE 1

| Metal (ppm) | Sample A | Sample B |
|---|---|---|
| Aluminum | 385 | 256 |
| Antimony | <11.2 | <16 |
| Arsenic | 0.213 | 0.156 |
| Barium | 43.2 | 26 |
| Beryllium | <1.35 | <2 |
| Bismuth | <7.52 | <16 |
| Boron | 82.2 | 63 |
| Cadmium | 0.450 | 0.295 |
| Calcium | 98,470 | 76,240 |
| Chromium | <1.80 | <8 |
| Cobalt | <0.75 | <2 |
| Copper | 12.6 | 8 |
| Iron | 7,900 | 4,500 |
| Lead | 0.751 | 0.389 |
| Lithium | <7.5 | <16 |
| Magnesium | 3,180 | 2,110 |
| Manganese | 103 | 60.5 |
| Mercury | <0.018 | <0.018 |
| Molybdenum | <1.68 | <2 |
| Nickel | 2.84 | 1 |
| Phosphorus | 1,890 | 1,250 |
| Potassium | 17,600 | 11,900 |
| Selenium | <23.6 | <24 |
| Silicon | 867.6 | 339 |
| Sodium | 2,490 | 2,120 |
| Strontium | 137 | 97 |
| Tellurium | <11.2 | <24 |
| Thallium | <7.52 | <16 |
| Tin | <4.49 | 3 |
| Titanium | 288 | 105 |
| Vanadium | 2.2 | 2 |
| Zinc | 32.1 | 16 |
| Zirconium | <10.1 | <16 |
| Sulfur | 1,370 | 2,910 |

As indicated in Table 1, the biochar produced showed levels of heavy metals, e.g., arsenic, cadmium, lead, and mercury, all below 1 part per million (ppm). Therefore, the biochar produced in accordance with various embodiments of this disclosure may be regarded as safe for use in food products.

The iodine number of a substance is indicative of the relative gas chemisorption (i.e., adsorption) ability of the subject material. The iodine number (the amount of iodine absorbed, in milligrams, by one gram of material) for the biochar produced in accordance with various embodiments of this disclosure was 90.10 for Sample A, and 154.0 for Sample B. These results are comparable to the iodine numbers of other biochars formed from biomasses comprising other materials. For example, biochar produced from biomass comprising cotton gin trash (e.g., burs and stems, immature cottonseed, lint, leaf fragments, and dirt (i.e., organic matter)) by pyrolysis at 700 degrees C. showed an iodine number of between 150 and 200 (Hernandez-Maglinao et al., Improving the Surface Areas and Pore Volumes of Bio-char Produced from Pyrolysis of Cotton Gin Trash via Steam Activation Process, INTERNATIONAL JOURNAL OF SCIENTIFIC ENGINEERING AND SCIENCE, Vol. 3 (6), 15-18 (2019)). As another example, biochar produced from woody biomass comprising, for example, wood chips, sawdust, bark, foliage, tree limbs, unmerchantable roundwood, and stumps, showed an iodine number of 142 for one sample and 82 for another sample (Andersen et al., A Comparison of Producer Gas, Biochar, and Activated Carbon from Two Distributed Scale Thermochemical Conversion Systems Used to Process Forest Biomass, ENERGIES, Vol. 6, 164-183 (2013)). Accordingly, the iodine numbers for the biochar produced in accordance with the embodiments of this disclosure are comparable to those associated with other preexisting biochars.

Table 2 shows a list of biochars formed from various feedstock sources (i.e., biomass compositions), and the compositional makeup of each biochar (Ippolito et al., Biochar elemental composition and factors influencing nutrient retention, BIOCHAR FOR ENVIRONMENTAL MANAGEMENT, pgs. 137-162 (2014)).

TABLE 2

| | C | N | P | K | S | Ca | Mg | Fe | |
|---|---|---|---|---|---|---|---|---|---|
| Biomass | (%) | (%) | (g kg⁻¹) | (g kg⁻¹) | (g kg⁻¹) | (g kg⁻¹) | (g kg⁻¹) | (g kg⁻¹) | Cu |
| Corn | 58.8 | 1.06 | 2.35 | 19.0 | 0.37 | 8.64 | 7.10 | 7.30 | 115 |
| Wheat/barley | 61 | 1.4 | —‡ | 1.26 | — | 12.6 | 9.88 | 1.94 | — |
| Rice straw/husk | 44 | 1.40 | 1.20 | 0.70 | 3.90 | — | — | — | — |
| Sorghum | 56 | 0.7 | 2.34 | 4.14 | — | — | — | — | — |
| Soybean stover | 75 | 1.6 | — | — | 0.40 | — | — | — | — |
| Peanut shell | 75 | 1.8 | 2.05 | 11.0 | 0.90 | 3.30 | 1.48 | — | — |
| Pecan shell | 76 | 0.3 | — | 116 | 0.20 | 6.00 | 0.59 | 0.04 | 34.0 |
| Hazelnut shell | 78 | 0.5 | 0.32 | 4.73 | — | 3.13 | 0.61 | —‡ | — |
| Switchgrass | 74 | 1 | 1.70 | 8.25 | — | 3.10 | — | 0.10 | 8.3 |
| Bagasse | 79 | 0.9 | 0.67 | 2.23 | — | 7.33 | 1.77 | 0.43 | — |
| Coconut coir | 74 | 0.9 | — | — | — | — | — | — | — |
| Food waste | 44 | 3.3 | 6.64 | 19.2 | — | 51.8 | 4.93 | — | — |
| Other (grass, leaves, orange | 65 | 1.2 | 1.62 | 14.4 | 1.30 | 5.92 | 3.31 | 1.35 | 66 |
| peel, other green wastes) | | | | | | | | | |
| Hardwoods | 74 | 0.7 | 1.14 | 9.47 | 15.6 | 10.1 | 9.53 | 1.80 | 4.8 |
| Softwoods | 75 | 0.8 | 0.74 | 16.9 | 0.23 | 20.7 | 18.0 | 9.64 | 1.4 |
| Papermill waste | 20 | 0.1 | 0.85 | 3.31 | — | 281 | 2.73 | — | — |
| Poultry manure/litter | 35 | 2.2 | 33.1 | 60.2 | 9.26 | 103 | 12.2 | 2.91 | 513 |
| Turkey manure/litter | 32 | 2 | 31.4 | 48.0 | 4.80 | 48.2 | 10.4 | 3.22 | 648 |
| Swine manure | 45 | 2.8 | 60.8 | 23.4 | 8.25 | 48.0 | 29.0 | 6.17 | 472 |
| Dairy manure | 58 | 2.4 | 8.59 | 17.2 | 2.70 | 26.9 | 11.8 | 5.87 | 107 |
| Cattle manure | 49 | 1.90 | 9.17 | 40.6 | 4.25 | 28.8 | 9.93 | 2.86 | 114 |
| Biosolids/sewage sludge | 24 | 1.1 | 42.4 | — | — | — | — | — | 222 |

As shown in Table 2, biochar from biomass comprising more plant-based materials (i.e., biomass originating from plants) has a relatively higher concentration of carbon, and a relatively lower concentration of phosphorus, nitrogen, potassium, sulfur, and calcium than biochar from biomass comprising animal-based materials (i.e., biomass originating from animals, such as manure).

The compositional make-up of a biochar may indicate the use for which such biochar is most suited. For example, all biochars may be suitable to serve as a soil conditioner to enhance soil quality (e.g., to increase soil organic carbon and/or organic matter content, to improve soil physical properties, such as moisture and/or minerals retention and holding capacity, and/or the like). Biochars having relatively higher carbon concentrations (e.g., biochars formed from plant biomass) may be more suited to serve as a soil conditioner. As another example, biochars having relatively higher concentrations of plant nutrients, such as phosphorus, nitrogen, potassium, and calcium (e.g., biochars formed from animal biomass, such as manure), may be more suited to serve as a plant-nutrient source (i.e., a fertilizer). However, to achieve desired characteristics regarding soil conditioning and fertilizing from a single biochar, a biochar may be created with balanced amounts of carbon and plant nutrients, rather than having significantly more of one than the other. For example, the biomass used to create a biochar may be a blend of different biomass types (e.g., a mix of animal-originating biomass and plant-originating biomass) to achieve a biochar with a desired compositional makeup to serve a certain purpose. As another example, different biochars may be blended together to achieve desired characteristics of the biochar mixture, such as blending biochar produced from animal-originating biomass with biochar produced from plant-originating biomass.

Table 3 shows the compositional make-ups (shown as the weight percent of each element) for a number of samples of biochar prepared in accordance with various embodiments of this disclosure. Particles 1-5 are particles from biochar Sample A in Table 1, and Particles 6-7 are particles from biochar Sample B in Table 1. Biochar produced in accordance with various embodiments of this disclosure shall be referred to as the “Subject Biochar(s).”

TABLE 3

| Element | | | | | | | |
|---|---|---|---|---|---|---|---|
| (wt. %) | Particle 1 | Particle 2 | Particle 3 | Particle 4 | Particle 5 | Particle 6 | Particle 7 |
| Carbon | 30.90 | 38.10 | 64.12 | 26.50 | 53.23 | 29.52 | 42.23 |
| Oxygen | 39.50 | 35.47 | 25.58 | 47.41 | 37.48 | 46.15 | 42.06 |
| Fluorine | 1.31 | ND | ND | ND | ND | — | — |
| Sodium | 0.38 | 0.14 | 0.13 | 0.33 | 0.42 | 0.27 | 0.39 |
| Magnesium | 0.45 | 0.52 | 0.16 | 0.78 | 0.25 | 0.34 | 0.26 |
| Aluminum | ND | ND | 0.05 | 0.25 | ND | 0.21 | ND |
| Silicon | 0.38 | 0.29 | 0.08 | 0.86 | 0.05 | 0.46 | 0.11 |
| Phosphorus | 0.28 | 0.54 | 0.11 | ND | 0.11 | — | — |
| Sulfur | 0.16 | 0.45 | ND | 0.06 | ND | 0.15 | 0.06 |
| Chlorine | 0.66 | 1.39 | 0.32 | 0.08 | 0.68 | 0.36 | 0.36 |
| Potassium | 2.28 | 6.47 | 1.87 | 0.26 | 1.25 | 1.21 | 2.28 |
| Calcium | 22.88 | 14.63 | 5.02 | 9.42 | 6.03 | 14.94 | 10.54 |
| Titanium | 0.29 | 1.40 | 2.56 | 3.46 | 0.51 | 1.69 | 1.42 |
| Iron | 0.54 | 0.61 | ND | 10.59 | ND | 4.71 | 0.30 |

The phosphorus and potassium values in the tested Subject Biochar were at levels indicating possible usefulness as a fertilizer or a fertilizer supplement or aid. The tested Subject Biochar samples in Table 3 had a range of phosphorus concentration of zero (none detected) to 0.54 weight percent, the average of which is greater than the weight percent of phosphorus of the biochar produced from plant-based biomass shown in Table 2. Similarly, the tested Subject Biochar samples in Table 3 had a range of potassium concentration of 0.26 to 6.47 weight percent, the upper end and average of which is greater than the weight percent of potassium of the biochar produced from plant-based biomass, and comparable to that of the biochar produced from animal-based biomass (manure), shown in Table 2. Similarly, the tested Subject Biochar samples in Table 3 comprise a relatively higher weight percentage of calcium. Therefore, the Subject Biochar may be suitable for providing plant nutrients (e.g., when mixed with soil), and/or may be more suitable for providing plant nutrients than biochars produced from plant-based biomass.

The Subject Biochar may also be suitable to serve to enhance soil quality. The tested Subject Biochar comprises a carbon concentration that is comparable or higher than the biochars listed in Table 2 produced from animal manure biomass. Therefore, the Subject Biochar may serve as a more effective soil conditioner than biochar produced from animal-originating biomass. Additionally, without being bound by theory, the Subject Biochar may comprise higher concentrations of various elements and/or compounds if produced from biomass at a higher temperature (higher than 620 degrees C., which produced the tested Subject Biochar), as literature indicates that pyrolysis temperatures higher than 620 degrees C. may yield better (i.e., more element/compound-rich) biochar.

Overall, the Subject Biochar, as indicated by the results for the tested Subject Biochar, may effectively act as a soil conditioner and/or a fertilizer, without the need for significant supplementation from another biochar produced from a certain biomass composition. However, for example, to enhance the effectiveness of the Subject Biochar in providing plant nutrients, the Subject Biochar may be supplemented with biochar produced from animal-originating biomass (or the biomass comprising cellulose acetate to create the Subject Biochar may be supplemented with animal-originating biomass). As another example, to enhance the effectiveness of the Subject Biochar in enhancing soil quality, the Subject Biochar may be supplemented with biochar produced from plant-originating biomass (or the biomass comprising cellulose acetate to create the Subject Biochar may be supplemented with plant-originating biomass). To achieve desired results and desired function of the biochar, the biomass used to produce biochar in accordance with various embodiments may comprise any suitable amount of cellulose acetate (e.g., about 20 percent weight cellulose acetate, about 30 percent weight cellulose acetate, about 40 percent weight cellulose acetate, about 50 percent weight cellulose acetate, about 60 percent weight cellulose acetate, about 70 percent weight cellulose acetate, about 80 percent weight cellulose acetate, about 90 percent weight cellulose acetate, about 100 percent weight cellulose acetate, wherein “about” used in this context means plus or minus 5 percent weight).

Along with the Subject Biochar being utilized as a soil supplement and/or fertilizer, the Subject Biochar may also be utilized as odor-reduction media, livestock feed supplement, and/or have an additional benefit of reducing CO₂ within the environment.

As a soil supplement, the Subject Biochar may improve water and nutrient retention of the soil. Therefore, soil comprising the Subject Biochar may be more resistant to drought. The porosity of the Subject Biochar (e.g., in the significant concentration of carbon therein), along with its composition of beneficial components therein (e.g., plant nutrients), may allow the Subject Biochar to attract and hold moisture, minerals, and provide a habitat for microorganisms valuable in facilitating the growth of other living organisms (e.g., vegetation). The iodine number of the tested Subject Biochar indicates the Subject Biochar's ability to chemisorb materials or compounds, discussed herein, and accordingly, its ability to perform such functions.

As a livestock feed supplement, the Subject Biochar may provide various benefits. For example, biochar added to livestock feed has been shown to improve digestion of the animals, increase immunity of the animals from disease, reduce chronic botulism, increase feed and energy efficiency (e.g., by improving energy adsorption by the animals from feed), increase growth rates of the animals, and/or reduce methane production. Without being bound by theory, the ability of the Subject Biochar to adsorb other materials (indicated by the iodine numbers of the tested Subject Biochar, discussed herein) may cause the Subject Biochar to attract and hold nutrients and microorganisms beneficial to facilitating animal function, as well as cause the Subject Biochar to absorb and/or adsorb and sequester harmful materials, microorganisms, or particulates (e.g., mycotoxins, pesticides, plant toxins, and/or toxic pathogens or metabolites). Therefore, the Subject Biochar being ingested by animals as a feed supplement may serve to mitigate or prevent the effects of poisoning or infection and/or the effects of harmful materials, microorganisms, or particulates ingested by such animals. In addition, adding biochar to litter which receives animal excrement may cause a decrease in the release of harmful gases from the litter and/or excrement (e.g., the release of ammonia gas from chicken excrement).

Similarly, the Subject Biochar may serve as a tool for dealing with manure (e.g., on farms, for example, in a manure lagoon), for example, for odor or gas emission management. Gases emitted from manure, such as methane, ammonia, nitrous oxide, and hydrogen sulfide, may be greenhouse gases and/or detrimental to air quality and the environment as a whole. Additionally, such gases may be toxic to humans and animals. The Subject Biochar may be applied to manure as a biocover to not only cover the odor of the manure, but also absorb or adsorb and sequester the gases emitted from the manure, thus at least partially neutralizing their potentially harmful effects. With the addition of the Subject Biochar's ability to attract and hold beneficial nutrients and microbes, as well as absorb or adsorb and sequester harmful materials or compounds, the Subject Biochar and/or manure may be repurposed after the Subject Biochar has acted as a biocover.

Method 200, and/or similar methods, may also serve as, or have the purpose of, an alternative to landfill disposal. The majority of biomass 110 used in the systems and methods described herein (e.g., cellulose acetate and/or cellulose acetate tow from, or made for use in, cigarette butts, cigarette filters, cigarette filter components, filter rods, and/or the like) is normally disposed (e.g., by applicable industry) in a landfill, or is litter on the ground. However, the systems and methods discussed herein may be used to obtain (from industry) and/or repurpose such biomass 110 to produce a stable carbon source such as the Subject Biochar. The Subject Biochar may have the benefits of, as discussed, increasing and improving agricultural output (e.g., as a fertilizer and/or soil enhancer), reducing odor in poultry, bovine, and/or swine operations, retaining water, reducing fertilizer rates, and/or and potentially reducing soil erosion. A further benefit of the Subject Biochar may result from the absorption or adsorption properties of the biochar when added to the soil and used to treat water to reduce harmful effects related to fertilizer runoff, heavy metals, and contaminants. The resultant products of syngas, oils, etc. may be converted into power, insecticides, or other commodities.

The Subject Biochar may be considered a precursor to activated carbon. Activated carbon is an advanced adsorbent that can be used to remove pharmaceutical compounds and/or other harmful materials or compounds (e.g., materials comprising heavy metals) from water sources or soil, for example. However, activated carbon is significantly more expensive to produce than biochar. The Subject Biochar's ability to attract and hold harmful materials (e.g., through the porous form and high surface area of the biochar) make the Subject Biochar a possible tool to removing such harmful materials from water and/or soil, for example. In various embodiments, biochar may be able to remove 90 percent of targeted pharmaceutical or other potentially harmful compounds from water.

Additionally, systems and methods described herein may comprise environmentally-neutral or environmentally-positive processes, and/or carbon-neutral or carbon-negative solutions. Without being bound by theory, the production of the Subject Biochar may release lesser amounts of greenhouse gases (e.g., carbon dioxide) than would be released otherwise by natural decomposition of the biomass. During natural decomposition of biomass, the materials of the biomass naturally release carbon (e.g., in carbon dioxide) that the biomass captured throughout its existence or lifetime. Pyrolysis of the biomass may also release carbon, but much of the carbon comprised in the biomass may be converted to a more stable form in the Subject Biochar resulting from the pyrolysis. Therefore, such carbon in the Subject Biochar may be shielded from being released as a greenhouse gas (e.g., carbon sequestration). The addition of the Subject Biochar to other materials that will degrade or decompose may also prevent the release of harmful gases from such degradation from decomposition (e.g., ammonia, carbon dioxide, methane, nitrous oxide, and/or the like). This ability of the Subject Biochar to sequester carbon and other compounds may cause the additional benefit of preventing or decreasing carbon emissions into the air or environment from the precursory biomass.

Overall, this disclosure allows thousands of tons of biomass (e.g., synthetic fiber cellulose acetate, such as cigarette filter waste) that otherwise would be considered waste, and left to decompose in a landfill or as litter (for years), to be removed from landfill and the environment as litter, degraded (e.g., through pyrolysis), and repurposed in one or more of the uses discussed herein, resulting in the significant benefits discussed.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods and apparatus are provided herein. In the detailed description herein, references to “one embodiment”, “an embodiment”, “various embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A method, comprising:
disposing biomass into a processing chamber;
regulating at least one of a temperature and a pressure in the processing chamber; and
processing the biomass to create biochar;
wherein the biomass comprises cellulose acetate.

2. The method of claim 1, wherein the processing chamber comprises a kiln.

3. The method of claim 2, wherein the processing chamber comprises a rotary kiln.

4. The method of claim 1, wherein an atmosphere in the processing chamber during the processing the biomass comprises little or no oxygen.

5. The method of claim 4, wherein the atmosphere in the processing chamber comprises a nonreactive gas.

6. The method of claim 1, wherein the biomass comprises between 50 and 100 percent by weight cellulose acetate.

7. The method of claim 1, wherein the cellulose acetate comprises a synthetic fiber cellulose acetate.

8. The method of claim 7, further comprising procuring the biomass comprising the synthetic fiber cellulose acetate from at least one of discarded cigarette filters and discarded cigarette filter components.

9. The method of claim 1, wherein the regulating the temperature comprises increasing the temperature in the processing chamber 10 degrees C. per minutes up to between 550 and 800 degrees C.

10. The method of claim 1, further comprising sizing the biomass prior to the disposing the biomass into the processing chamber.

11. The method of claim 10, wherein the sizing the biomass is completed via a shredder.

12. The method of claim 1, wherein the biomass is disposed into the processing chamber from a feed system comprising a feeder valve.

13. The method of claim 1, further comprising discharging an exhaust from the processing chamber through an exhaust system; and scrubbing the exhaust in a scrubber comprised in the exhaust system.

14. The method of claim 1, further comprising discharging the biochar into a discharge system; and cooling the biochar.

15. The method of claim 1, further comprising supplementing the biomass comprising cellulose acetate with biomass comprising at least one of animal manure or a plant-originating material.

16. The method of claim 1, further comprising utilizing the biochar as at least one of a soil supplement, a fertilizer, an animal feed supplement, or an odor reducer.

17. A biochar formed by pyrolyzing biomass comprising cellulose acetate.

18. The biochar of claim 17, wherein the cellulose acetate comprises synthetic fiber cellulose acetate.

19. The biochar of claim 17, wherein the biomass comprises between 50 and 100 percent by weight cellulose acetate.

20. The biochar of claim 17, wherein the biomass further comprises at least one of animal manure or a plant-originating material.
